(19) United States
(12) Patent Application Publication
(10) Pub. No.: US 2005/0226821 A1
(43) Pub. Date: Oct. 13, 2005

(54) Cosmetic formulations containing l-arginine oligomers

(76) Inventors: Jacob Waugh, Palo Alto, CA (US); Michael Dake, Stanford, CA (US); Christopher J. Elkins, Palo Alto, CA (US); Pamela N. Cifra, Hillsborough, CA (US)

Correspondence Address: Morgan & Finnegan, 3 World Financial Center, New York, NY, 10281-2101, US

(21) Appl. No.: 10/505,299

(22) PCT Filed: Feb. 21, 2003
(86) PCT No.: PCT/US03/05399

§ 371 Date: Aug. 20, 2004

Related U.S. Application Data
(60) Provisional application No. 60/358,879, filed on Feb. 22, 2002.

(51) Int. Cl. A61K 38/10; A61K 38/08; A61K 7/28; A61K 7/42; A61K 7/06; A61K 7/11

(52) U.S. Cl. 424/50; 514/14; 514/15; 514/16; 424/70.14; 424/59

(57) ABSTRACT

Oligomers of L-arginine may be used to provide prophylactic or therapeutic cosmetic or other enhancements to keratinous tissues such as skin, hair, lips and gums through vasodilation. Topical compositions preferably comprise (a) an enhancing effective amount of an oligomer having from 7 to 15 subunits, each subunit consisting of a member of the group selected from L-arginine and physiologically acceptable salts of L-arginine that enhance vasodilation through production of nitric oxide, and (b) a cosmetically or dermatologically acceptable vehicle.

### BACKGROUND

L-arginine is a naturally occurring amino acid, which participates in many important biochemical reactions essential to the normal physiology of an organism. This amino acid is found in most proteins consumed in our daily diet and can be metabolized to support glucose synthesis or catabolized to produce energy. In addition to L-arginine's important biochemical roles, it has also been found to be the immediate precursor of the endogenous vasodilator substrate found in the arterial blood vessels termed endothelial-derived relaxing factor (EDRF), which is chemically identical to nitric oxide (NO). Nitric oxide is present in macrophages and thought to mediate a number of cytotoxic actions.

Present in all cells in the body, NO, formed from L-arginine by nitric oxide synthase (NOS), is believed to be essential in homeostatic processes. Normally, NO has a very short half-life—only 3-5 seconds—due to rapid inactivation by oxyhemoglobin in the formation of methemoglobin. Modifications in the function of NO within the cardiovascular system have been associated with numerous diseases that seem to stem from endothelial cells lining blood vessels (von der Leyen et al., “Gene therapy inhibiting neointimal lesion in vivo transfer of endothelial cell nitric oxide synthase gene,” Proc Natl Acad Sci USA, 92(4): 1137-1141 (1995)). Endothelial cells may produce locally acting paracrine factors that favor vasodilatation, inhibition of cell proliferation and maintenance of blood fluidity, which are all characteristics of a healthy vascular system. NO is one of the factors that exhibit these effects (von der Leyen et al., “Gene therapy inhibiting neointimal lesion in vivo transfer of endothelial cell nitric oxide synthase gene,” Proc Natl Acad Sci USA, 92(4): 1137-1141 (1995)).

In highly vascularized tissues such as lips, gums, genitalia, etc, vasodilatation leads to transient, reversible increases in tissue mass and sensitivity. A method for enhancing vasodilatation in these tissues would therefore lead to a tissue with enlarged appearance for the duration of vasodilatation—a limiting factor for current products. For all skin, cutaneous vasodilatation creates a natural blush appearance and can enhance superficial skin temperature. Additionally, appearance of certain fine lines and wrinkles might be lessened, leading to additional cosmetic benefits. For cosmetic purposes, enhanced lip size has become desirable. Natural appearing changes in skin and lip color have also become important drivers for cosmetics. For highly innervated structures such as genitalia, these same changes will lead to increased sensitivity to stimulation as well as increased turgor. Additionally, structures such as gums regress with age and with inappropriate care (hard toothbrush bristles, etc). This regression is still thought to be irreversible. However, the appearance might be mitigated by vasodilatation for at least a transient cosmetic benefit.

Research has also shown that NO from NO donors or modification of nitric oxide synthesis upregulates the expression of VEGF (vascular endothelial growth factor) in vascular smooth muscle cells (Jozkowicz et al., “Genetic augmentation of nitric oxide synthase increases the vascular generation of VEGF,” Cardiovascular Res, 51:773-783 (2001)). Vascular endothelial growth factor is of major importance for skin vascularization. Expression of VEGF is increased in hyperplastic epidermis of psoriasis (Detmar et al., “Overexpression of vascular permeability factor/vascular endothelial growth factor and its receptors in psoriasis,” J Exp. Med., 180:1141-1146 (1994)), in wound healing (Brown et al., “Expression of vascular permeability factor (vascular endothelial growth factor) by epidermal keratinocytes during wound healing,” J Exp. Med., 176:1375-1379 (1992)), and in other skin diseases characterized by enhanced angiogenesis (Brown et al., “Increased expression of vascular permeability factor (vascular endothelial growth factor) in bullous pemphigoid, dermatitis herpetiformis, and erythema multiforme,” J. Invest. Dermatol., 104:744-749 (1995); Brown et al., “Overexpression of vascular permeability factor (VPFNEGF) and its endothelial cell receptors in delayed hypersensitivity skin reactions,” J. Immunol., 154:2801-2807 (1995)). The hair follicle undergoes distinct cyclic expansion and regression that leads to rapidly changing needs for its vascular support. Adequate supply of blood is a prerequisite for normal cell growth and differentiation. The hair follicle undergoes a life-long cyclic transformation. There are three phases of the hair growth cycle: anagen, catagen and telogen. The anagen phase is involved with rapid proliferation of follicular keratinocytes and elongation and thickening of the hair (Yano et al., “Control of hair growth and follicle size by VEGF-mediated angiogenesis,” The J. Clin. Invest., 107:409-417 (2001)). After anagen is completed, the hair enters the catagen phase. In the catagen phase, the matrix cells gradually stop dividing and eventually keratinize. This phase is short and usually lasts about 2-3 weeks. When full keratinization is achieved, the hair enters the last phase of the cycle, telogen. During the telogen (resting) phase, keratinized hair falls out, and a new matrix is gradually formed from the stem cells in the basal layer of the outer epithelial root sheath bulge (Jankovic et al., “The control of hair growth,” Dermatology Online Journal, 4(1), 2). Afterwards, a new hair starts to grow and the follicle is back in the anagen phase.

Research findings demonstrate that pronounced angiogenesis occurs during murine hair follicle cycling (Yano et al., “Control of hair growth and follicle size by VEGF-mediated angiogenesis,” The J. Clin. Invest., 107:409-417 (2001)). Overexpression of VEGF in follicular keratinocytes resulted in accelerated hair regrowth and in increased sized of hair follicles (Yano et al., “Control of hair growth and follicle size by VEGF-mediated angiogenesis,” The J. Clin. Invest., 107:409-417 (2001)). This result provides first direct evidence that promotion of angiogenesis can promote hair growth and also hair thickness. However, VEGF-induced vascular permeability appears dependent on both NO production and prostaglandin production (Murohara et al., “Vascular endothelial growth factor/vascular permeability factor enhances vascular permeability via nitric oxide and prostacyclin,” Circulation, 97:99-107 (1998)). In mice lacking the eNOS gene, impaired angiogenesis was not improved by administration of VEGF, which suggests that eNOS is downstream from VEGF (Murohara et al., “Nitric oxide synthase modulates angiogenesis in response to tissue ischemia.,” J. Clin. Invest., 111:2567-2578 (1998)). Endothelial nitric oxide synthase is a downstream mediator for in vivo angiogenesis (Murohara et al., “Nitric oxide synthase modulates angiogenesis in response to tissue ischemia.,” J. Clin. Invest., 111 :2567-2578 (1998)). Promoting eNOS activity by administration of L-arginine accelerates in vivo angiogenesis (Murohara et al., “Nitric oxide synthase modulates angiogenesis in response to tissue ischemia.,” J. Clin. Invest., 111:2567-2578 (1998)). Therefore, NO may help to promote hair regrowth and increased size of hair follicles.

Nitric oxide is a vasodilator that broadly inhibits DNA synthesis and limits cell proliferation. However, NO diffuses freely and cannot be stored in any topical base and so cannot be delivered topically. An approach to this problem would be to use L-arginine, but studies have shown that the amino acid does not cross skin effectively and also cannot be applied topically.

### SUMMARY OF THE INVENTION

It has now been found that oligomers of L-arginine may be used to provide cosmetic and other enhancements to keratinous tissues such as skin, hair, lips and gums through vasodilation.

In one embodiment this invention comprises a topical composition for enhancement of keratinous tissues such as skin, lips, hair, or gums comprising (a) an enhancing effective amount of an oligomer having from 7 to 15 subunits, each subunit consisting of a member of the group selected from L-arginine and physiologically acceptable salts of L-arginine that enhance vasodilation through production of nitric oxide, and (b) a cosmetically or dermatologically acceptable vehicle.

In another embodiment, this invention comprises a method of prophylactically or therapeutically caring for the skin, hair, lips or gums comprising applying thereto an enhancing effective amount of the above composition.

These and further embodiments are described in more detail below.

### DETAILED DESCRIPTION OF THE INVENTION

This invention relates to compositions and methods for cosmetic and other enhancement of keratin tissues such as skin, hair, lips and gums by applying a cosmetically or dermatologically acceptable composition that contains an enhancing effective amount of an oligomer of L-arginine (also referred to as “oligoarginine”).

Oligoarginine complexes have been shown to carry some agents across skin when covalently bound (Kown et al., “L-arginine polymer mediated inhibition of graft coronary artery disease after cardiac transplantation,” Transplantation, 71:1542-1548 (2001); Wender et al., “The design, synthesis and evaluation of molecules that enable or enhance cellular uptake: Peptoid molecular transporters,” Proc Natl Acad Sci USA, 97(24): 3003-13008 (2000)). For example, cyclosporin linked to a heptamer of arginine has been shown to penetrate the skin effectively (Rothbard et al., “Conjugation of arginine oligomers to cyclosporin A facilitates topical delivery and inhibition of inflammation,” Nat Med, 6:1253-1257 (2000)) [see also U.S. Pat. No. 6,306,993]. Numerous drugs are available for the treatment of primary cutaneous disease. However, these drugs are only effective when given systemically and are ineffective when administered topically because of poor absorption. in any case, what has been shown in the above-cited literature is that oligomers of arginine have been shown to provide delivery of other therapeutic agents. In contrast, the present invention is directed to the use of arginine oligomers as prophylactic or therapeutic/cosmeceutical agents in their own right, in treating keratinocyte tissues.

By “arginine oligomers” or, as described above, a polymer that contains arginine-type subunits, is meant a polymer that contains from 7 to 15, preferably 7, 9, 11, 13 or 15, more preferably 7, 9, 11 or 13, and most preferably 7 or 9 subunits, each subunit consisting of the amino acid L-arginine or of a physiologically acceptable salt of L-arginine that enhances keratin tissues by enhancing vasodilation through production of nitric oxide.

In one embodiment, the arginine oligomer consists solely of arginine. In another embodiment, however, it may be included in a somewhat larger polymer or peptide that contains other amino acids (e.g., glycine), providing that (a) such other amino acids are not therapeutically effective and (b) the arginine oligomer is situated at either the C-terminus or the N-terminus of the polymer or peptide. One example of such a peptide is gly₃-arg₇.

Suitable salts of L-arginine that may be used in these oligomers or polymers are those that are physiologically acceptable, i.e. salts that may be used in contact with skin, lips, hair or gums without causing any undesirable or deleterious effects. Examples of suitable salts include, but are not limited to, L-arginine hydrobromide, hydrochloride, sulfate, bisulfate, nitrate, acetate, oxalate, valerate, oleate, palmitate, stearate, laureate, borate, benzoate, lactate, phosphate, tosylate, citrate, maleate, fumarate, succinate, tartrate, naphthylate mesylate, glucoheptonate, lactiobionate and laurylsulfonate salts and the like. The salts may include alkali or alkaline earth metal cations, such as sodium, lithium, potassium, calcium, magnesium and the like, as well as nontoxic ammonium, quaternary ammonium and amine cations such as ammonium, tetramethylammonium, tetraethylammonium, methylamine, dimethylamine, trimethylamine, triethylamine, ethylamine and the like. (See, for example, Berge S. M. et al., “Pharmaceutical Salts,” J. Pharm. Sci., 66:1-19 (1977) which is incorporated herein by reference.) Among salts, the hydrochloride salt is preferred.

The L-arginine oligomers (both those forms that are oligomers containing solely L-arginine and those forms in which the L-arginine is included in a larger peptide) may also be provided in the form of derivatives that serve as sources of the L-arginine oligomers, including derivatives in which the oligomer is linked or anchored to a polymer (with or without degradable linkages) or to a hydrophobic, hydrophilic, or amphipathic compound, for instance, a polyol.

Compositions of the present invention may be used for a number of cosmetic or dermatological treatments or purposes. Hair care treatments include application of topical oligoarginine formulations to prevent hair loss or to promote hair regrowth on the scalp, or to increase the length and/or thickness of eyelashes and/or eyebrows. Compositions may be applied to lips for purposes such as improving the cosmetic appearance of lip contour and/or lip color, or for expansion of lip plumpness. Topical applications may be made to skin to reduce the appearance of wrinkles and fine lines, to reduce the appearance of excess tissue around the eyes (i.e., puffiness), to provide a natural blush appearance and effect to skin, or for other cosmetic benefits. Compositions of this invention may also be used to provide increased sensitivity, for example to increase genital sensation or turgor or to provide hand or foot warming.

Compositions may also be applied for dental purposes, e.g., to reduce the appearance of gum regression, or to induce gum regeneration.

The compositions of this invention are preferably in the form of products to be applied to the lips, the eyes, the skin, or the gums of humans. They therefore contain a cosmetically or dermatologically acceptable vehicle or medium, i.e. a vehicle or medium that is compatible with the tissues to which they will be applied.

The term “cosmetically or dermatologically acceptable,” as used herein, means that the compositions or components thereof so described are suitable for use in contact with these tissues without undue toxicity, incompatibility, instability, allergic response, and the like.

In addition, compositions of the invention may comprise any ingredient conventionally used in the fields under consideration, and particularly in cosmetics and dermatology.

The term “effective amount” as used herein means an amount of a compound, composition or oligomeric arginine according to this invention that is sufficient to significantly induce a positive benefit, preferably a positive skin, lip, hair, etc., appearance, or a sensory benefit, but that implicitly is a safe amount, i.e. one that is low enough to avoid serious side effects.

In practice, compositions of this invention include various types of compositions and formulations typically used for cosmetic and other care of the skin, hair, lips and gums and include, for instance:

skin-care preparations, e.g. skin-washing and cleansing preparations, soapless detergents, body lotions, emulsions, or skin oils, and preparations for cleansing and caring for blemished skin, e.g. peeling or scrub preparations or peeling masks;

bath preparations, e.g. liquid (foam baths, milks, shower preparations) or solid bath preparations, e.g. bath cubes and bath salts;

cosmetic personal care preparations, e.g. facial make-up in the form of day creams or powder creams, face powder, rouge or cream make-up;

eye-care preparations, e.g. eyeshadow, mascara, eyeliner, or eye creams;

lip-care preparations, e.g. lipsticks, lip gloss, or lip contour pencils;

nail-care preparations, such as nail polish and nail vanish;

foot-care preparations, e.g. foot baths, foot powders, foot creams or foot balsams, special deodorants and antiperspirants or callous-removing preparations;

light-protective preparations, such as sun milks, lotions, creams and oils, sun blocks or tropicals, pre-tanning preparations or after-sun preparations; skin-tanning preparations, e.g. self-tanning creams;

dental-care and mouth-care preparations, e.g. toothpastes or tooth powders and pastes, rinses or gels for gum treatment; and

hair-treatment or hair-care preparations, e.g., shampoos, conditioners, styling creams, styling gels, hair rinses, foams, hairsprays, or hair dyes and colorants.

In terms of their physical form, compositions of this invention may include solutions, emulsions (including microemulsions), suspensions, creams, lotions, gels, waxy products such as lipstick, powders, and solids of various types.

Such compositions will contain, in addition to the oligomeric arginines of this invention, other ingredients typically used in such products, such as antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, hair pigment additives, humectants, mica, minerals, polyphenols, silicones or derivatives thereof, sunblocks, vitamins, and phytomedicinals.

In addition to the oligomeric arginines, the compositions may contain other active ingredients used in skin, lip, hair or dental care. For instance, they may contain anti-acne actives; anti-wrinkle, anti-skin atrophy or skin repair actives; skin barrier repair actives; non-steroidal cosmetic soothing actives; artificial tanning agents and accelerators; sunscreen actives; sebum stimulators; sebum inhibitors; anti-oxidants; protease inhibitors; skin tightening agents; anti-itch ingredients; desquamating enzyme enhancers; anti-glycation agents; and mixtures of such actives.

In one embodiment or application, compositions of the present invention are useful for regulating signs of skin aging, particularly visible and/or tactile discontinuities in skin texture associated with aging. “Regulating the signs of skin aging” includes prophylactically regulating and/or therapeutically regulating one or more of such signs (similarly, regulating a given sign of skin aging, e.g., lines, wrinkles or pores, includes prophylactically regulating and/or therapeutically regulating that sign). As used herein, prophylactically regulating such signs includes delaying, minimizing and/or preventing signs of skin aging. As used herein, therapeutically regulating such signs includes ameliorating, e.g., diminishing, minimizing and/or effacing signs of skin aging.

By “signs of skin aging” is meant outward visibly and tactilely perceptible manifestations as well as other macro or micro effects due to skin aging. These signs include the development of textural discontinuities such as wrinkles, including both fine superficial wrinkles and coarse deep wrinkles, skin lines, crevices, bumps, large pores, scaliness, flakiness and/or other forms of skin unevenness or roughness, loss of skin elasticity, sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

Such signs may be induced or caused by intrinsic factors or by extrinsic factors, e.g., chronological aging and/or environmental damage. It should be noted, however, that the present invention is not limited to regulation of the above mentioned signs of skin aging which arise due to mechanisms associated with skin aging, but is intended to include regulation of said signs irrespective of the mechanism of origin.

The present invention is especially useful for therapeutically regulating visible and/or tactile discontinuities in mammalian skin texture, including texture discontinuities related to skin aging. This includes ameliorating, e.g., diminishing, minimizing and/or effacing visible and/or tactile discontinuities in the texture of mammalian skin, to thereby provide improved skin appearance and/or feel, e.g., a smoother, more even appearance and/or feel. For example, the length, depth, and/or other dimension of lines and/or wrinkles may be decreased, the apparent diameter of pores may decrease, or the apparent height of tissue immediately proximate to pore openings may decrease so as to approach that of the interadnexal skin.

The present invention is also useful for prophylactically regulating visible and/or tactile discontinuities in mammalian skin texture, including texture discontinuities associated with skin aging, that is, delaying, minimizing and/or preventing visible and/or tactile discontinuities in the texture of skin, to thereby provide improved skin appearance and/or feel, e.g., a smoother, more even appearance and/or feel.

The compositions of the present invention may also be used for stabilization or remodeling of hypodermal or deeper fat. Fat stabilization, particularly in humans, is generally associated with the appearance of aging attributed to fat atrophy as well as fat regression in the skin. The methods and compositions described herein can assist in preventing the formation of wrinkles and aid in ameliorating the appearance of deep wrinkles by supporting vascularity of the skin.

The following represent examples of compositions and uses of such compositions in accordance with this invention. However, these are presented solely as examples, and are not intended to limit the scope and nature of this invention in any way.

### EXAMPLES

### Example 1

### Topical Enhancement of Hair Growth (Eyelash, Eyebrow or Scalp)

A preparation of (L-arg)-(L-arg)-(L-arg)-(L-arg)-(L-arg)-(L-arg)-(L-arg)-(L-arg)-(L-arg) or [LR9] was obtained from Sigma Genosys at >70% purity. To achieve a therapeutic benefit on hair growth, a 10× stock of LR9 was prepared as 70 mg in 80 μl PBS. To 0.2 ml of Cetaphil moisturizer base was added the following:
LR9: 20 μl of 10× LR9 stock
Control: 20 μl PBS

After addition of LR9/PBS to moisturizer base, samples were mixed to homogeneity and stored at 4C overnight. C57 black six mice at 8 weeks of age were anesthetized with 3% isoflurane by inhalation, were shaved, and underwent depilation at mid-scapular dorsal region of 2 cm×2 cm with a rosin mixture (Surgiwax, Ardell, Commerce, Calif.) to induce the synchronized adolescent first hair cycle. Moisturizer was applied daily at 0.2 cc per day for either LR9 treatment or control for a fourteen day period at n=4 per group. After 14 days application the treated skin segment was harvested en bloc and subdivided into three equal portions: a cranial portion, a left lateral portion and a right lateral portion. The cranial portion and the left lateral portion were fixed in 10% neutral buffered formalin for 12-16 hours, then rinsed in 70% ethanol and embedded in paraffin. The right lateral portion was snap frozen in OCT medium at the time of harvest and stored at −35C until use.

Paraffin-embedded specimens were sectioned at 4-6 microns, deparaffinized, and stained with a combination of Verhoeff elastica stain and the Masson trichrome stain for morphological assessment of follicle area and number. Frozen samples underwent random hair pulls to determine hair shaft length. All procedures and analyses were performed by blinded observers. High resolution digital micrographs of each preparation were obtained using a Diagnostic Instruments SPOT camera (Diagnostic Instruments, Sterling Heights, Mich.) as displayed on a Nikon E600 epifluorescence microscope with plan-apochromat lenses. Images were analyzed using Image Pro Plus software (Media Cybernetics, Silver Spring, Md.) to determine total cross-sectional follicle area, follicle number, or hair length. Mean and standard error were assessed using Statview (Abacus Concepts, Berkeley, Calif.), with comparisons made using ANOVA repeated measures and significance determined at 95% with post-hoc testing using Fisher PLSD or Scheffe F-test.

Results are as follows:

Hair Shaft Length (pixels [1 pixel (for length)=2.774 microns], 14 days)

| Group: | Mean: | Std. Error: |
|---|---|---|
| LR9 | 1612.424 | 93.743 |
| control | 1131.009 | 60.44 |
| Comparison | anova (95) | |
| LR9 vs. control | ** | |

** = significant by fisher PLSD and Scheffe F-test (P = 0.0001)

Follicle Area (square pixels [1 square pixel (for area)=7.69 square microns], 14 days)

(by total x-sectional area)

| Group: | Mean: | Std. Error: |
|---|---|---|
| LR9 | 53546.00 | 3204.922 |
| control | 32458.333 | 2477.525 |
| Comparison | anova (95) | |
| LR9 vs. control | ** | |

** = significant by fisher PLSD and Scheffe F-test (P = 0.0001)

Number of follicles per cross-section (14 days)

| Group: | Mean: | Std. Error: |
|---|---|---|
| LR9 | 406.417 | 15.314 |
| control | 304.5 | 22.626 |
| Comparison | anova (95) | |
| LR9 vs. control | ** | |

** = significant by fisher PLSD and Scheffe F-test (P = 0.0001)

Thus, in the present example, LR9 was shown able, without any additional transdermal delivery platform, to statistically significantly enhance hair growth as measured by hair shaft length, total follicle area, and number of hair follicles.

### Example 2

### Topical Lip Color and Contour Enhancement

A preparation of (L-arg)-(L-arg)-(L-arg)-(L-arg)-(L-arg)-(L-arg)-(L-arg)-(L-arg)-(L-arg) or [LR9] was obtained from Sigma Genosys at >70% purity. To achieve a therapeutic benefit on hair growth, a 1× stock of LR9 for lip enhancement was prepared as 35 mg in 50 μl pbs, which was added to 2.0 ml of Revlon Lip Gloss base for the following groups:
LR9
Control (base only)

After addition of LR9 to lip gloss base, samples were mixed to homogeneity and stored at room temperature overnight. After baseline image acquisition using a Minolta Dimage 7 digital camera set to 105 macro (1:1) with standard lighting and scaling conditions, human subjects underwent topical application of LR9 or control gloss. All participants were blinded with respect to treatment or control application. Additional digital micrographs were acquired at time 0, 1, 5, 10, 15, and 30 minutes after lip gloss application. The gloss was then removed and participants asked to record duration of any lip contour or color changes. Additionally, any complications (irritation, etc) were also tracked for each. Each subject was treated at different timepoints with both groups (treatment or control). Image analysis of total photographic area was performed for each group, with care taken to normalize each to scales included at the time of photograph acquisition. Baseline images (pre-application) were verified to have statistically similar values between pre-LR9 gloss and pre-base gloss applications for each participant. Mean and standard error across timepoints 1, 5, 10, 15, and 30 minutes were determined for each participant after LR9-gloss or base gloss application. Values for each as well as photos acquired at matching timepoints are presented by participant below for 3 participants.

[image]

| | Mean Area | Standard |
|---|---|---|
| | (sq. in.) | Error |
| Base Gloss | 6.784 | 0.027 |
| LR9 | 7.745 | 0.283 |

[image]

[insert photos]

| | Mean Area | Standard |
|---|---|---|
| | (sq. in.) | Error |
| Base Gloss | 6.562 | 0.163 |
| LR9 | 7.411 | 0.128 |

[image]

| | Mean Area | Standard |
|---|---|---|
| | (sq. in.) | Error |
| Base Gloss | 7.523 | 0.055 |
| LR9 | 7.911 | 0.066 |

No participants noted any local irritation or other complications after administration of base gloss or LR9 gloss.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A composition for enhancement of keratinous tissues comprising (a) an enhancing effective amount of a polymer having from 7 to 15 subunits, each subunit consisting of a member of the group selected from L-arginine and physiologically acceptable salts of L-arginine that enhance vasodilation through production of nitric oxide, and (b) a cosmetically or dermatologically acceptable vehicle.

2. A composition according to claim 1 in which the polymer contains 7, 9, 11, 13 or 15 subunits.

3. A composition according to claim 1 in which the polymer contains 7, 9, 11, or 13 subunits.

4. A composition according to claim 1 in which the polymer contains 7 or 9 subunits.

5. A composition according to claim 1 selected from skin care compositions, lip care compositions, hair care compositions, and dental care compositions.

6. A composition according to claim 1 selected from creams, lotions, solutions, suspensions, powders, emulsions, gels, waxy products, powders and solids.

7. A composition according to claim 1 selected from skin-washing and skin-cleansing preparations, soapless detergents, body lotions, emulsions, skin oils, peeling or scrub preparations, peeling masks, foam baths, bath milks, shower preparations, bath cubes, bath salts, facial make-up eyeshadow, mascara, eyeliner, eye creams, lipsticks, lip gloss, lip contour pencils, nail polish, nail varnish, foot baths, foot powders, foot creams, foot balsams, callous-removing preparations, sun milks, sun lotions, sun creams, sun oils, sun blocks, pre-tanning preparations, after-sun preparations, self-tanning creams, toothpastes, tooth powders, gum treatment pastes, gum treatment gels, gum rinses, shampoos, conditioners, styling creams, styling gels, hair rinses, foams, hairsprays, hair dyes and hair colorants.

8. A composition according to claim 1 in which the subunits are selected from L-arginine and physiologically acceptable salts of L-arginine.

9. A composition according to claim 1 in which the subunits are selected from L-arginine and L-arginine salts selected from the group consisting of L-arginine sulfate, hydrobromide, hydrochloride, sulfate, bisulfate, nitrate, acetate, oxalate, valerate, oleate, palmitate, stearate, laureate, borate, benzoate, lactate, phosphate, tosylate, citrate, maleate, fumarate, succinate, tartrate, naphthylate, mesylate, glucoheptonate, lactiobionate and laurylsulfonate; alkali and alkaline earth metal salts of L-arginine; and ammonium, quaternary ammonium and amine salts of L-arginine.

10. A composition according to claim 1, further containing one or more ingredients selected from antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, hair pigment additives, humectants, mica, minerals, polyphenols, silicones or derivatives thereof, sunblocks, vitamins, and phytomedicinals.

11. A composition according to claim 1, further containing one or more additional skin care, lip care, dental care or hair care active agents.

12. A composition according to claim 11 further containing one or more ingredients selected from anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; 5-alpha reductase inhibitors; desquamating enzyme enhancers; anti-glycation agents; and mixtures thereof.

13. A composition according to claim 1 in which the polymer further comprises a hydrophobic, hydrophilic, or amphipathic moiety, or a second polymer, linked or anchored to a terminal L-arginine subunit of the polymer.

14. A composition for enhancement of keratinous tissues comprising (a) an enhancing effective amount of a polymer having from 7 to 15 contiguous L-arginine subunits, each such subunit consisting of a member of the group selected from L-arginine and physiologically acceptable salts of L-arginine that enhance vasodilation through production of nitric oxide, the polymer also containing one or more amino acids other than L-arginine, providing that (i) such other amino acids are not therapeutically effective and (ii) the contiguous L-arginine subunits are situated at either the C-terminus or the N-terminus of the polymer; and (b) a cosmetically or dermatologically acceptable vehicle.

15. A composition according to claim 14 in which the polymer contains 7, 9, 11, 13 or 15 contiguous L-arginine subunits.

16. A composition according to claim 14 in which the polymer contains 7, 9, 11, or 13 contiguous L-arginine subunits.

17. A composition according to claim 14 in which the polymer contains 7 or 9 contiguous L-arginine subunits.

18. A composition according to claim 14 selected from skin care compositions, lip care compositions, hair care compositions, and dental care compositions.

19. A composition according to claim 14 selected from creams, lotions, solutions, suspensions, powders, emulsions, gels, waxy products, powders and solids.

20. A composition according to claim 14 selected from skin-washing and skin-cleansing preparations, soapless detergents, body lotions, emulsions, skin oils, peeling or scrub preparations, peeling masks, foam baths, bath milks, shower preparations, bath cubes, bath salts, facial make-up eyeshadow, mascara, eyeliner, eye creams, lipsticks, lip gloss, lip contour pencils, nail polish, nail varnish, foot baths, foot powders, foot creams, foot balsams, callous-removing preparations, sun milks, sun lotions, sun creams, sun oils, sun blocks, pre-tanning preparations, after-sun preparations, self-tanning creams, toothpastes, tooth powders, gum treatment pastes, gum treatment gels, gum rinses, shampoos, conditioners, styling creams, styling gels, hair rinses, foams, hairsprays, hair dyes and hair colorants.

21. A composition according to claim 14 in which the L-arginine subunits are selected from L-arginine and physiologically acceptable salts of L-arginine.

22. A composition according to claim 14 in which the L-arginine subunits are selected from L-arginine and L-arginine salts selected from the group consisting of L-arginine sulfate, hydrobromide, hydrochloride, sulfate, bisulfate, nitrate, acetate, oxalate, valerate oleate, palmitate, stearate, laureate, borate, benzoate, lactate, phosphate, tosylate, citrate, maleate, fumarate, succinate, tartrate, naphthylate, mesylate, glucoheptonate, lactiobionate and laurylsulfonate; alkali and alkaline earth metal salts of L-arginine; and ammonium, quaternary ammonium and amine salts of L-arginine.

23. A composition according to claim 14, further containing one or more additional skin care, lip care, dental care or hair care active agents.

24. A composition according to claim 23 further containing one or more active compounds selected from anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; 5-alpha reductase inhibitors; desquamating enzyme enhancers; anti-glycation agents; and mixtures thereof.

25. A composition according to claim 14, further containing one or more ingredients selected from antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, hair pigment additives, humectants, mica, minerals, polyphenols, silicones or derivatives thereof, sunblocks, vitamins, and phytomedicinals.

26. A composition according to claim 14 in which the polymer further comprises a hydrophobic, hydrophilic, or amphipathic moiety, or a second polymer, linked or anchored to a terminal L-arginine subunit of the polymer.

27. A method of prophylactically or therapeutically caring for the skin, hair, lips or gums comprising applying thereto an enhancing effective amount of a composition according to claim 1.

28. A method according to claim 27 in which the composition is applied topically.

29. A method according to claim 27 in which the composition is applied to promote angiogenesis in hair follicles.

30. A method according to claim 27 in which the composition is applied to alleviate signs of aging in skin.

31. A method according to claim 27 in which the composition is applied to enhance the appearance of lips.

32. A method according to claim 27 in which the composition is applied to enhance sensitivity of skin.

33. A method according to claim 27 in which the composition is applied for stabilization or remodeling of fat.

34. A method according to claim 27 in which the composition is applied for treatment of gum regression.

36. A method of prophylactically or therapeutically caring for the skin, hair, lips or gums comprising applying thereto an enhancing effective amount of a composition according to claim 14.

37. A method according to claim 36 in which the composition is applied topically.

38. A method according to claim 36 in which the composition is applied to promote angiogenesis in hair follicles.

39. A method according to claim 36 in which the composition is applied to alleviate signs of aging in skin.

40. A method according to claim 36 in which the composition is applied to enhance the appearance of lips.

41. A method according to claim 36 in which the composition is applied to enhance sensitivity of skin.

42. A method according to claim 36 in which the composition is applied for stabilization or remodeling of fat.

43. A method according to claim 36 in which the composition is applied for treatment of gum regression.
